(19) United States
(12) Patent Application Publication
(10) Pub. No.: US 2010/0281935 A1
(43) Pub. Date: Nov. 11, 2010

(54) SYSTEM FOR PROCESSING ORGANIC WASTE

(75) Inventor: Rune Brandal, Ulsteinvik (NO)

Correspondence Address: DENNISON, SCHULTZ & MACDONALD, 1727 KING STREET, SUITE 105, ALEXANDRIA, VA, 22314, US

(73) Assignee: GLOBAL ENVIRO INTERNATIONAL AS, Selje (NO)

(21) Appl. No.: 12/675,141

(22) PCT Filed: Aug. 20, 2008
(86) PCT No.: PCT/NO2008/000296

§ 371 Date: Jul. 14, 2010

(30) Foreign Application Priority Data
Aug. 29, 2007 (NO) 20074401

(51) Int. Cl. C12M 1/33; C05F 11/08

(52) U.S. Cl. 71/8; 435/290.4

(57) ABSTRACT

System for processing organic was including several separate elements, in which organic waste is embedded at one end of the system, and mature compost is removed from the other end. The separate elements include at the least one grinding mill, one dehumidifier and one compost machine. The system also includes means for adding water before the dehumidifier, and means for ensuring that the liquid leaving the dehumidifier has a temperature above about 25° C., and preferably above 30° C. The invention also relates to a method for producing fertilizer, in which organic waste is ground and supplied with water, the fat- and liquid fractions are separated at a temperature above 25° C., and preferably above 30° C., and the solid mass is dehumidified and composted.

The present invention relates to a system for processing organic waste, especially food waste from institutional households such as institutions, restaurants and/or boats and ships. The invention also relates to a method for producing compost with excellent fertilizer properties.

### BACKGROUND

Today, institutional households produce very large quantities of food waste; both left-overs from the kitchen and left-overs from the guests. This waste must be transported for further treatment, and demands large resources both internal and external. When food waste should be stored, it must be taken reservations to ensure that it does not contaminate new foodstuff, and does not come in conflict with the production of new food. This requires large areas, and efforts to reduce smell/overheating of the waste. It is decreed by law that food waste should be stored in refrigerator rooms, but the waste is often stored without refrigeration, facing the corresponding problems of vermins, such as mice and rats. There will also be a risk that persons handling the waste get infected either by the food waste or the vermins related to storage without refrigeration. Besides, the transport of the food waste is very environmental unfriendly, as it is normally done by car, and often over large distances.

Processing of food waste is normally combustion, and thus energy, nourishment and trace elements are released into the surroundings with small chances of recycling. In order to reduce the amount of food waste, it may be put in a compost machine. A compost machine for an institutional household will, however, demand a lot of space, and it is not appropriate to place it in the kitchen. The waste must thus be stored temporarily and then moved to the compost machine. Further, not all waste can be put directly into the compost machine, such as whole bones and the similar. Traditional aerobic processing of food waste, such as in a compost machine, provides compost products which may be used as soil conditioner, but has little fertilization effect. As a rule, processes having short-time composting have large development of organic acids, which is inhibiting to germination and plant growth.

Another problem is that production of ecological foodproducts often leads to an unbalance between supplied amount of fertilizer and nutrient in sold crop. Most ecological growing systems have negative nutrient balance; they consume the nutrients of the soil. Farmers working ecological, have few real opportunities to better the nutrient balance, without returning nutrients. Thus, it is desirable to make compost having good fertilizer effect and which contains important nutrients, in such a way that the amount of crop and the nutrient balance are maintained in ecological cultivation systems.

### OBJECT

The main object of the present invention is to obtain a system for handling organic waste, preferably from institutional households, reducing the amount needing to be transported away, minimizing the need for intermediate storage, and minimizing the risk for directly infection to humans. The waste should not be handled in such a way that it might contaminate food being, or going to be, prepared. Further, it is an object that the waste should be processed to compost having good fertilizer effect.

### THE INVENTION

The object is met by an invention according to the characterizing part of the independent claims. Further advantageous features are stated in the corresponding, dependent claims.

The invention relates mainly to a system comprising several separate elements, where waste is embedded in one end of the system, and mature compost may be take out in the other end. The waste is first grinded finely in a grinding mill with water supply, then fat and water is separated from protein and carbohydrate rich residue in a dehumidifier, and finally the protein and carbohydrate rich residue is composted in a compost machine. The fat is preferably separated from the water in a fat collector, and the water may either be led into the pipeline network or be led back into the system.

The separate main elements of the invention comprises at the least a grinding mill with water supply, a dehumidifier, and a compost machine, and preferably a fat collector. In a specially preferred embodiment, the system also comprises a transport system, preferably based on use of vacuum, for transporting the waste internally in the system.

As used herein, “dehumidifier” is meant to describe any device removing liquid from solids, and removing so much liquid that the amount of solids become over 30%, preferably over 50%.

The inlet to the grinding mill is preferably placed close to where food waste is produced, normally in or in proximity of a kitchen. The grinding mill grinds the waste finely, and the grinding mill should be of a type capable of finely grinding all organic waste, including bones. The inlet to the grinding mill may be designed with water supply in such a way that plates and the similar may be rinsed off, and that the waste is led into the grinding mill by the water flow. In some cases it might be advantageous with several waste points, meaning several grinding mills at a distance from each other. All the grinding mills may, however, lead to the same dehumidifier and compost machine, in such a way that the system in total does not increase substantially.

Before the grinded waste is led into the dehumidifier, it must be added water, and the temperature of the waste must be sufficiently high to melt the fat. In order to make sure that the fat is removed together with the water, the liquid leaving the dehumidifier has a temperature above 25° C., preferably above 30° C. This may be performed in many ways, preferably is the waste supplied with water having a temperature above 25° C. before the dehumidifier, and it is especially preferred if this water is added via nozzles in the grinding mill. When water is added before the actual mill of the grinding mill, the food waste will be grinded with hot water present, and this will contribute to better dissolution of the fat.

The system is performed with means to ensure that the liquid leaving the dehumidifier has a temperature above 25° C., preferably above 30° C. These means may be the construction of the system; that the time period between supply of hot water and discharge from the dehumidifier is so short that the water does not chill sufficiently before it leaves the dehumidifier. In these cases, the temperature in the leaving liquid is measured at the start-up of the system, whereafter the temperature of the supplied water is adjusted. In other cases, the temperature of the leaving water is measured continuously, and the heat supply adjusted in order to ensure that the leaving liquid maintains a temperature above 25° C., preferably above 30° C.

In case the temperature is below 25° C., additional heat must be added to the dehumidifier, either by adding hotter water, or in another way. If the temperature of the liquid leaving the dehumidifier drops below 25° C., the fat will set and stay together with the protein and carbohydrate rich material, which again will influence the fertilizer properties of the prepared fertilizer, very negative. Food waste will contain several different types of fat, having different melting point, and some types have a melting point above 30° C. When the liquid holds a temperature above 30° C., most of the fat will be removed with the water, and the fat staying with the protein and carbohydrate rich material will constitute such a small part of the mature compost that it does not get influenced.

The dehumidifier may be of any type being design to separate liquid and grinded waste, and which removes so much liquid that the content of total solids is more than 30%, preferably more than 50%. One example of a dehumidifier, is a feed screw placed inside a perforated pipe, in such a way that the liquid is forced out through the perforations. The liquid being removed in the dehumidifier is preferably led through a fat collector, and possibly a sludge remover, before it is led out on the regular sewer system. In the fat remover, the fat is removed from the water, and may either be transported away, or be used as biofuel. It may also be used a sludge remover to remove the particles from the water, possibly the fat and sludge remover may be a combined unit. The water may either be led out on the sewer system, or be recycled back to the system, in order to reduce the total water consume. In case the water should be recycled, it must, of course, be filtered and/or purified first.

The dehumidified pulp is led from the dehumidifier to a compost machine. It is used a compost machine having heat and air supply, and stirring, in such a way that the grinded, dehumidified pulp can be transformed to compost during a short period of time. Further, the compost machine should be designed for continuously supply and the size must be adjusted to the amount of waste being produced. In a preferred embodiment of the compost machine, the mature compost will be pushed forward in the machine and taken out via an outlet, for instance an output screw.

The compost made according to the present invention, may be used as soil conditioner having excellent fertilizer effect, and must thus be sanitationed. This may be performed in many ways, both as a part of the compost machine and as a finishing process, which will be obvious to a person skilled of the art.

The transport of the waste internal in the system; from grinding mill to dehumidifier, and from dehumidifier to compost machine, may be based on gravity, or the system may comprise a transport system. Such a transport system may be based on pumps, screws or preferably, vacuum. If such a transport system is used, it will be advantageous to have a storage tank underneath the grinding mill, and that the transport system is run only at intervals.

A system according to the present invention may advantageously both be post-installed in existing buildings, and integrated in new buildings.

### EXAMPLES

In the following, will preferred embodiments of the separate main elements of the invention; grinding mill, dehumidifier and compost machine, be described. These embodiments are examples and should not be interpreted as limiting to the invention.

### Grinding Mill

A preferred embodiment of a grinding mill for use according to the present invention, comprises principally an inlay lid, a grinding unit and a flushing system. The inlay lid is relatively large and leads directly into the grinding unit. The grinding mill is designed in such a way that the grinding stops if the inlay lid is opened. It is not possible to start the grinding until the lid is in place, and it is also performed with a mechanical locking system. Besides, the engine working the grinding is performed with brakes, in order to stop immediately if the lid is opened. The system may possibly be performed in such a way that the lid cannot be opened until the grinding has stopped, with a time delay if desired. This locking system is performed to prevent injuries, and may be performed in many ways, which will be obvious for a person skilled of the art.

From the inlay lid to the grinding unit, a least a lower part of the walls slant towards each other, in such a way that the waste being embedded in the grinding mill is led down towards the grinding unit. The bottom of the grinding unit comprises a rotating disk, performed with teeth on the outer edge. The parts of the wall being adjacent to the rotating disk, are also performed with teeth, and when the disk rotates the waste will be grinded between these teeth. In the following, the interaction between the teeth of the rotating disk and the teeth of the walls will be referred to as a “tearing rim”. The teeth, both on the rotating disk and especially on the walls, may have different grades of fineness in vertical direction, in such a way that the waste first gets coarsely ground and then finely ground when it passes through the tearing rim. The waste must be grinded to such a degree of fineness that all fat may be dissolved with added water, preferably to particles <4 mm.

In order to further assist the grinding of the waste, it is advantageous to install knives to the rotating disk, wherein the knives cut the waste entering the grinding mill into smaller parts. The knives are preferably installed in such a way that they protrude a distance from the disk, and thus that the waste is already being coarsely cut when it reaches the tearing rim.

To ensure that all of the waste passes through the tearing rim, and does not remain on top of the rotating disk, it may advantageously be installed angular “guiding rails” to the disk, which in addition to tear the waste apart, pushes bones and the similar towards the tearing rim. The walls may also have different ribs or elevations, protruders and similar to guide the waste down towards the rotating disk.

In principle, the gravity should ensure that the waste comes down into the grinding unit and through the tearing rim. In cases where the gravity is not sufficient, or it is desirable to ease the process, it may advantageously be installed at the least a couple of water nozzles in the grinding mill. These will also contribute to a better cleaning, and the grinding gets more easily. When water is added before the grinding, the fat will more easily melt and be mixed with the water. The first nozzle should preferably be installed underneath the inlay lid, at the upper edge of the tapering part. The water from this nozzle will ensure that possible residues clinging to the walls towards the grinding unit, will be flushed down, and that the grinding gets more easily. The other nozzle should preferably be mounted underneath the tearing rim in such a way that it flushes towards the rim. The water from this will contribute to leading grinded waste out of the grinding unit, and thus contribute to cleaning and continuity of the system. In order to reduce the total amount of water added to the system, the nozzles may advantageously be run at intervals.

In alternative embodiments, the system may be performed with other means for adding water having a temperature above 25° C., and thus, the temperature of the water in the nozzles will not be important for anything but cleaning. In other alternative embodiments water IS added to the waste before the dehumidifier, and the waste is heated before it is lead into the dehumidifier.

The grinding mill may, above the inlay lid, be performed with a hand flusher, such as a commercial kitchen flusher, to flush plates, pans and the similar, and to flush the grinding mill itself, for cleaning.

The grinding mill is in its entirety, performed of stainless steel, and without unnecessary joints and welds. This is to avoid irregularities to the surfaces, which may give positive conditions for growth of bacteria.

### Dehumidifier

A preferred embodiment of a dehumidifier for use according to the present invention, comprises a buffer tank, wherein the grinded waste from the grinding mill is supplied. In the buffer tank the waste from the grinding mill is stored and led into the dehumidifier in a continuously and suitable amount. The dehumidifier may also work without the buffer tank, but then it must be dimensioned to handle the largest amount of waste being produced over a short period of time. With the buffer tank, the amount of waste can be scattered over a larger period of time, and the dehumidifier can be dimensioned to handle an average amount of produced waste. As mentioned, it is a premise in order to remove fat, that the liquid leaving the dehumidifier has a temperature above 25° C., preferably above 30° C., and if necessary, the buffer tank may be performed with heating elements.

In a preferred embodiment the dehumidifier comprises a feed screw placed in a perforated pipe. The perforated pipe, with the feed screw, is preferably slanting, in such a way that the inlet for pulp to be dehumidified, is below the outlet. The pulp to be dehumidified is pressed between the feed screw and the perforated pipe, and the liquid seep through the perforations. The increasingly dryer pulp is transported up towards the outlet of the dehumidifier. The density and angle of the screw windings, and the slanting of the dehumidifier must be adjusted to each other, in such a way that optimal dehumidification is reach during shortest possible time.

For further dehumidification it is an advantage that the feed screw, in its upper part, does not have screw windings. The pulp will thus only be moved by pulp being added from below, and a small counter pressure will arise, pressuring out more liquid.

To prevent that the perforations in the surrounding pipe clutters up, the screw windings are preferably performed with brushes at their outer edge. These brushes bear against the inside of the pipe and will brush off eventual pulp. A further advantage is to place a number of nozzles at the lower edge of the pipe, flushing the perforations from the outside. The water being used for flushing has a temperature above 25° C., preferably above 30° C.

The dehumidifier is preferably run at intervals. When the pulp has passed through the dehumidifier, it will be an advantage to let the feed screw run a short period, in order to have the inside of the pipe sufficiently brushed, and that the nozzles may flush the perforations clean. If it is continuous supply of grinded waste from the grinding mill, the dehumidifier should nevertheless be run at intervals with cleaning in between. This is to ensure that the perforations of the pipe does not clutter up and prevent the liquid from leaving the pulp.

The liquid leaving the dehumidifier should, preferably, be lead through a fat remover, and possibly a mud remover, before it is led into the sewer system, or recycled to the process. The dehumidified pulp is led to a compost machine.

### Compost Machine

A preferred embodiment of a compost machine comprises two chambers with heating elements, turning devices and air-circulation system. The inlet for pulp to be composted is placed in one end of the first chamber, and the outlet for mature compost is placed at the opposite end of the other chamber. The chambers are separated with a partition wall in which the upper part is constituted of a grid.

The heating elements in the compost machine make sure that the temperature is maintained at a level being suitable for composting and drying. The turning devices are performed and placed in such a way that they continuously or at intervals, turn the pulp, and this results both in that the composting process turns aerobe and that the pulp being the most composted and driest, will be on top of the pulp. The pulp is moved towards the second chamber by incoming pulp, and will be pushed through the grid and thereby enter the second chamber. The wall between the chambers has only grid in the upper part, and thus only the driest pulp will get through the grid. The partition wall with grid, prevents that wet and/or clogged pulp gets close to the outlet, and ensures thus that all pulp added to the compost machine gets composted.

Close to the inlet it is placed an exhaust fan, drawing gas/air out of the compost machine, and which preferably is so powerful that a small negative pressure arises in the whole compost machine. Close to the outlet for mature compost it is placed an air inlet, in such a way that the air circulating in the compost machine is countercurrent with the pulp to be composted. The air being drawn in and the air removed from the compost machine may preferably be heat exchanged in order to avoid unnecessary heat loss. It is also an advantage that the air being removed from the compost machine is led through different filters in order to avoid undesired smell. However, the air circulation system may be performed in many ways, which will be obvious for a person skilled of the art.

### EXAMPLES

The invention will in the following, be described with reference to the enclosed Figures, showing a preferred embodiment of the invention.

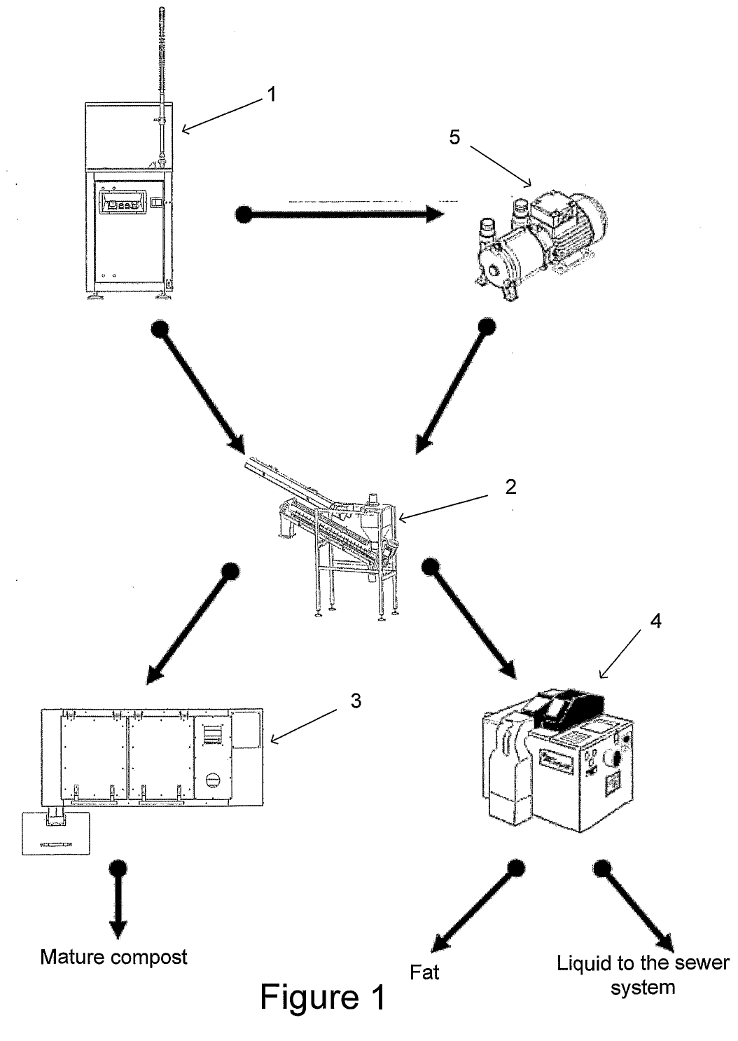

FIG. 1 shows the different elements put together in the system according to the invention,

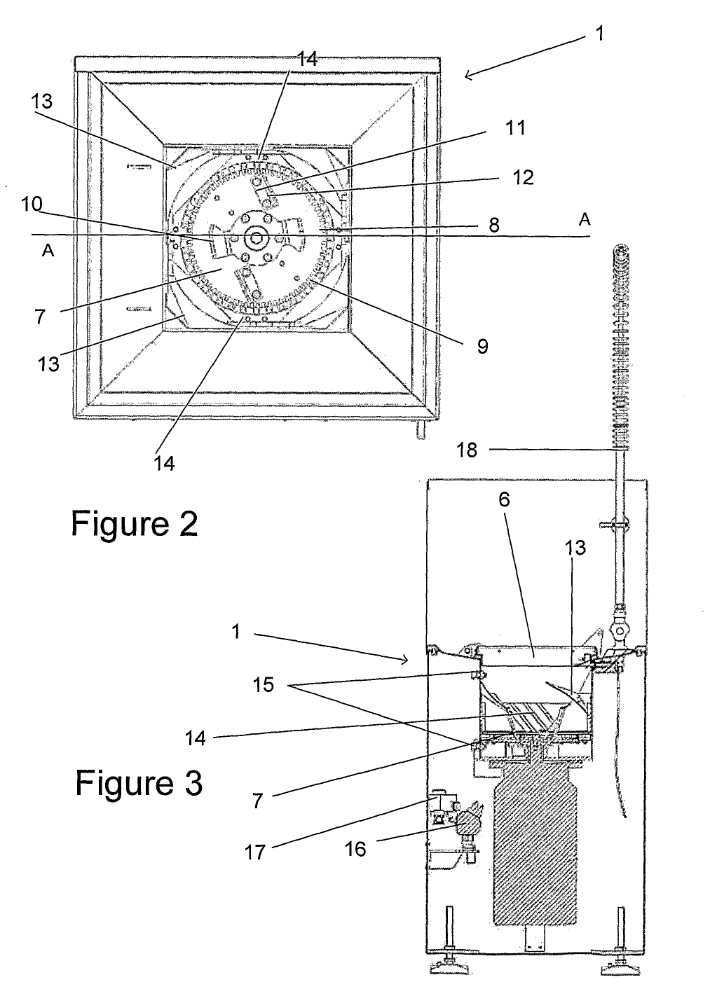

FIG. 2 shows a grinding mill according to the invention, seen from above,

FIG. 3 shows a vertical section along line A-A, of the grinding mill in FIG. 2,

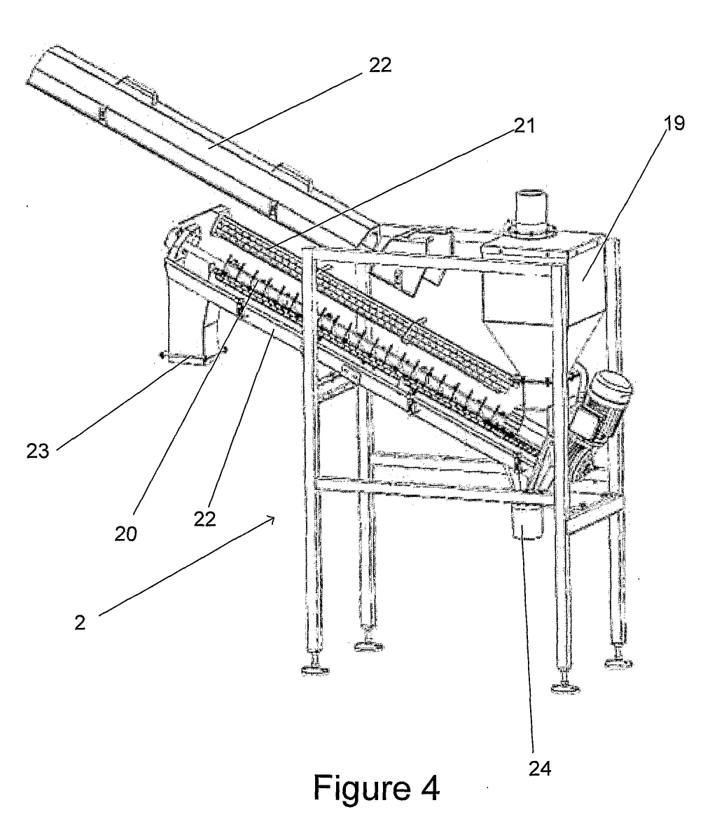

FIG. 4 shows the dehumidifier in partly exploded sketch,

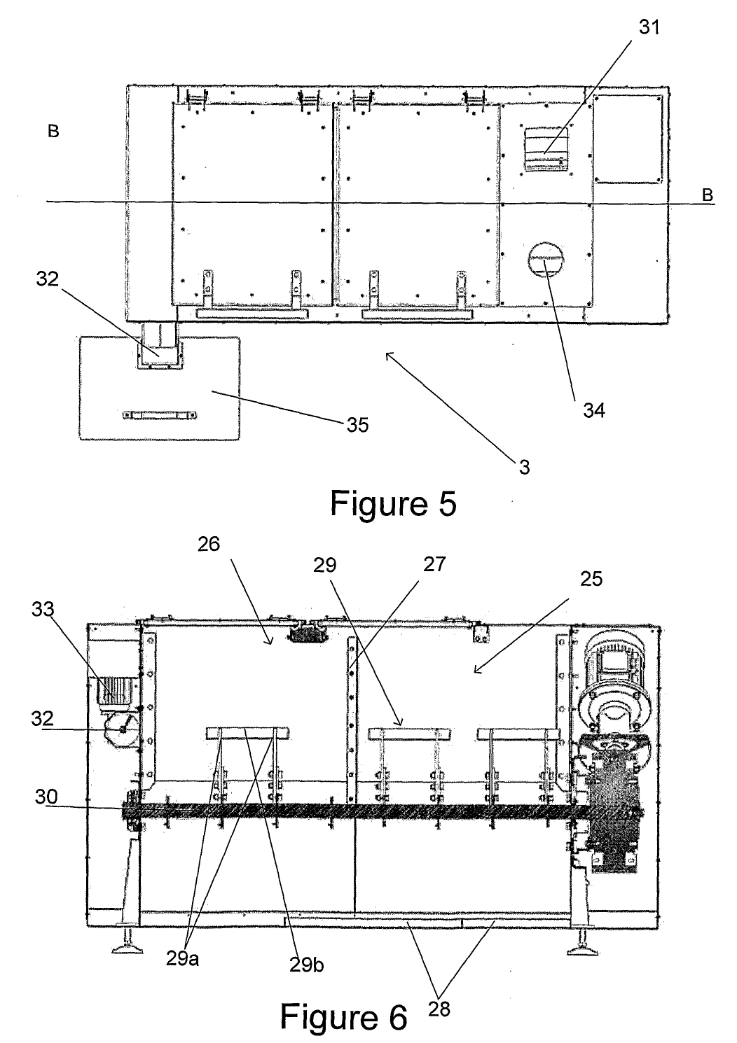

FIG. 5 shows the compost machine from above, and

FIG. 6 shows a vertical section along line B-B of FIG. 5.

In FIG. 1 the system according to the present invention, is shown, as the different elements are placed in order. Organic waste is added to a grinding mill 1, gets grinded, supplied with water having a temperature above 30° C., and led to a dehumidifier. In the dehumidifier liquid is removed from the pulp which then is led to a compost machine 3, while the liquid removed in the dehumidifier is led to the sewer system via a sludge/fat remover 4. It is also drafted an alternative comprising a pump 5, helping the transport of the pulp from the grinding mill to the dehumidifier, for instance by vacuum.

The grinding mill shown in FIGS. 2 and 3 is performed with an inlay lid 6 over tapering sides slanting down towards the grinding unit. FIG. 2 shows the grinding unit from above, and the inlay lid is removed for better overview. The grinding unit comprises a rotating disk 7 being performed with horizontal teeth 8 at its outer edge, and the adjacent wall of the grinding mill is provided with corresponding teeth 9. When the rotating disk 7 rotates, the teeth 8, 9 will go against each other, and constitute thus a tearing rim. Further, the rotating disk is provided with knives 10 coarsely cutting the waste to be grinded, before the waste gets down to the tearing rim. On the disk it is also installed a couple of guiding rails 11 guiding the waste to be composted towards the tearing rim, and leading bones and the similar into it. In the shown, especially preferred embodiment, the guiding rails have an uneven upwardly edge 12 contributing to coarsely cutting of the waste.

In the upper part of the shown embodiment of the grinding mill, it is further installed four slanting wings 13, pressuring/guiding the waste down towards the grinding unit. Besides, in the tapering part it is designed a number of slanting ribs 14 on the inside. In an especially preferred embodiment these ribs slant counter to the rotation direction of the rotating disk 7, in such a way that they tear up the rotation of the waste, and create turbulence. This contributes to improving the coarse cutting of the waste by the knives 10 in the centre, and besides they inhibit the waste from clinging to the walls of the grinding mill. Moreover, the ribs 14 will stiffen the walls of the grinding mill, and thus contribute to less noise.

The grinding mill further comprises two water nozzles 15, one above the tapering part of the grinding mill, and one underneath the tearing rim. These nozzles add water having a temperature above 30° C., and provide for both cleaning, as waste does not get stuck on the walls but flushed further in the system, and for washing out the fat.

Besides, in the shown embodiment is the grinding mill provided with a mixing battery 16 of itself, in such a way that the temperature of the water to the nozzles 15 may be regulated, and a time set valve 17 regulating the intervals whereby the flushing should take place. It is an especially advantage in periods where extra fat food is served, that the temperature of the water may be adjusted up, and that the flushing intervals can be increased. It will be obvious for a person skilled of the art that both the valve and the battery easily can be moved out of the grinding mill, and that corresponding functions may be achieved in another way. The shown commercial kitchen shower 18 may also be replaced by other means to achieve the same function.

FIG. 4 shows a dehumidifier 2 corresponding to the present invention, in partly exploded sketch. The dehumidifier 2 comprises a buffer tank 19 leading down onto a feed screw 20 enclosed by a perforated pipe 21. The perforated pipe 21 is enclosed by a cover 22 both with regard to security and to collect liquid being pressed out of the pulp. The grinded pulp is led to the feed screw 20 via the buffer tank 19, and led upwards of the feed screw 20 at the same time as liquid is pressed out through the perforations of the pipe 21. Further, the feed screw 20 itself, is not provided with screw windings at the outlet end, in order to press out maximal amount of liquid, as explained above. The dehumidified pulp is led out of the dehumidifier via an outlet 23 in the upper end, and further to the compost machine 3. In addition, the dehumidifier has an outlet 24 for liquid, as the liquid is led out to the sewer system, possibly via a sludge/fat remover 4. As said above, the liquid may also be recycled back into the system. In a preferred embodiment is the dehumidifier 2 equipped with nozzles (not shown) at the lower edge of the perforated pipe 21, in such a way that the pipe can be flushed and thereby kept clean.

FIGS. 5 and 6 show the compost machine 3 from above and in cross section, respectively. The compost machine comprises a chamber being divided in two 25, 26, heating element 28, turning devices 29 and air circulation system. The chamber is divided in two by a partition wall; as the upper part of the wall is a grid 27. Turning devices are placed in both chambers, and in the preferred shown embodiment, they are designed as two vertical arms 29a with a horizontal bar 29b inbetween. The vertical arms 29a are fastened on a rotating axle 30, and in this way the horizontal bar 29b is led through the pulp and turns it. In an especially preferred embodiment several, preferably three, turning devices are placed on the axle 30, at about 60 degrees distance, in order to turn the pulp further.

The grinded, dehumidified pulp is led to the inlet 31 of the compost machine in the one end of the first chamber 25 of the compost machine 3, and the mature compost can be taken out at the outlet 32 on the opposite end of the second chamber 25. The air circulation system circulates air countercurrent of the pulp, in such a way that the inlet for air is by the outlet 32 of the mature compost, and the outlet 34 for the air is by the inlet for the pulp. It is an advantage to have a fan and/or pump (not shown) performing the air circulation. In the shown preferred embodiment the heating elements 28 ensures sufficient temperature, and the air circulation system ensures removal of moisture, in such a way that the composting time can be reduced.

The outlet for mature compost 32 is preferably placed some distance up on the wall, in such a way that only the top layer of the pulp in the compost machine can come into the outlet. Further, it is an advantage that the outlet is a feed screw rather than an open hole, because it reduces the risk of clogging. In the shown embodiment the mature compost is stored temporarily in a container 35 on the outside of the machine. The fan/pump at the outlet of the air circulation system is so powerful that the whole of the compost machine 3, including the container 35, is kept under gently vacuum.

After the composting is finished, it is an advantage that the compost is sanitationed, to ensure that possible bacteria and/or fungus do not go with the compost further. The sanitation may be performed as a part of the compost machine, or it can be performed in a separate finishing step. It will preferably be used heat, but this may be performed in many ways, which will be familiar to a person skilled of the art.

The sludge/fat remover and the pump possibly transporting the pulp between the different elements of the system, can be of any type, which will be obvious to a person skilled of the art. Thus, they are not described any further in this example.

### Analysis of Compost Produced According to the Present Invention

Mixed food waste was embedded into the system, and after 18 hours the compost was mature in the compost machine. The amount of mass was reduced by about 90%. About 10 liters of water was added to 8 kg waste and the contents of total solids after the dehumidifier was about 50%.

The compost was analyzed by AnalyCen AS (Moss, Norway), and the contents are stated in the table below.

| Parameter | Method | Unit | | |
|---|---|---|---|---|
| pH | AS4 | | | 6.1 |
| Conductivity | | mS/m | | 1820 |
| Total solids | AS5 | % | | 85.1 |
| Loss on ignition | AS5 | g/100 | g TS | 78.5 |
| Total org. carbon | AJ31 | g/100 | g | 51.2 |
| Kjeldahl-N | AS6 | g/100 | g TS | 4.2 |
| C/N-ratio | | | | 12 |
| Ammonium-N | AS7 | mg/100 | g TS | 116 |
| Nitrate-N + Nitrite-N | AS8 | mg/100 | g TS | 1.7 |
| Phosphorus | AS1 | g/100 | g TS | 0.63 |
| Potassium | AS1 | g/100 | g TS | 0.65 |
| Calcium | AS1 | g/100 | g TS | 4.2 |
| Magnesium | AS1 | g/100 | g TS | 0.15 |
| Sodium | AS1 | mg/100 | g TS | 710 |
| Sulphur | AS1 | mg/100 | g TS | 340 |
| pH in soil | | | | 6.0 |
| Phosphorus | AL | mg/100 | g | 458 |
| Potassium | AL | mg/100 | g | 505 |
| Magnesium | AL | mg/100 | g | 142 |
| Calcium | AL | mg/100 | g | 3410 |
| Sodium | AL | mg/100 | g | 510 |

Samples of soil mixed with compost from the present invention showed considerable amounts soluble phosphorus (P-AL), and the compost is thus a considerable source of phosphorus. Further, the ratio between nitrogen and phosphorus corresponds with the needs of many plants. Further, most of the nitrogen is bound in organic form, and the share of mineral N (ammonium-N and nitrate-N) is small. The C/N ratio of the compost is 12, corresponding to cultured soil, and suggests that considerable amounts of mineral N will be released when the compost is degraded in soil.

The AL method is a declaration method for organic fertilizer, and when looking at the ration between total content and AL soluble content (ammonium-lactate and acetic acid) of P, K, Mg, Ca and Na in the compost from the present invention, the solubility is high. The high solubility suggests that the product contains plant nutrients which can be utilized by plants.

The method for producing compost according to the present invention, is novel and different from traditional production because one removes fat and water, and only the protein and carbohydrate rich residue are processed to compost. In traditional production of compost from food waste it is made organic acids resulting in low pH, and the compost may thus not be used directly as fertilizer. Further, the organic acids bind the nutrients in such a way that plants cannot utilize them. Another advantage with compost produced according to the present invention is the low C/N number. This compost has a C/N number of 12, which corresponds with the C/N number of cultivated soil, and the uptake in plants is thus assumed to be corresponding as for cultivated soil. Corresponding numbers for traditionally produced compost is ca 20.

pH in compost made according to the present invention is 6,1, which suggests that it is not made organic acids. Compost produced according to the present invention also contains few heavy metals but relatively high concentrations of soluble plant nutrients, and is thus suited as fertilizer both in ecological and traditional agriculture. Since ecological agriculture has few fertilizer alternatives, such treated food waste will be especially relevant. It will be well suitable to deliver compost made according to the present invention back to farms delivering food/farming product to the kitchen in question. Such a situation will represent an efficient recycling of nutrients with short distances.

1. System comprising several separate elements, wherein organic waste is embedded in one end of the system, and mature compost is taken out in the other end, characterized in that the separate elements comprise at the least one grinding mill (1), one dehumidifier (2) and one compost machine (3), and in that it is arranged means for adding water before the dehumidifier, and means for ensuring that the liquid leaving the dehumidifier has a temperature above ca 25° C., preferably above 30° C., and in that the liquid being removed in the dehumidifier is led through a fat collector before it is reused or led out on the regular sewer system.

2. System according to claim 1, characterized in that the water is added via nozzles (15) in the grinding mill (1).

3. System according to claim 2, characterized in that the nozzles (15) are placed above the grinding unit and below the rotating disk (7).

4. System according to claim 1, characterized in that the means to ensure that the liquid leaving the dehumidifier has a temperature above 25° C., preferably above 30° C., comprises a temperature gauge and a regulatory system.

5. System according to claim 1, characterized in that the means to ensure that the liquid leaving the dehumidifier has a temperature above 25° C., preferably above ca 30° C., comprises an adjusted installation of the different units.

6. System according to claim 1, characterized in that the system further comprises a transport system, preferably by vacuum, for transporting pulp internally in the system.

7. System according to claim 1, characterized in that the dehumidifier (2) comprises a feed screw (20) and a perforated pipe (21), wherein the feed screw (20) is placed inside the perforated pipe (21).

8. System according to claim 7, characterized in that the feed screw (20) contains brushes outermost on the screw windings, and that these brushes touch the inside of the perforated tube (21).

9. System according to claim 7, characterized in that the dehumidifier (2) further comprises nozzles for cleaning the perforated pipe (2).

10. System according to claim 1, characterized in that the compost machine (3) comprises devices (28) for heat supply, organs for turning the pulp, and an air-circulating system, wherein the air preferably runs countercurrent with the pulp to be composted.

11. Method for producing fertilizer, characterized in that organic waste is ground, supplied with water, the fat- and liquid fraction are separated at a temperature above 25° C., preferably above 30° C., and further that the solid mass is dehumidified and composted.

12. Method according to claim 11, characterized in that the fat-fraction is separated by adding water having a temperature above 25° C., preferably above 30° C., to the ground waste, and that the pulp thereafter is dehumidified.
